(19) United States
(12) Patent Application Publication
(10) Pub. No.: US 2026/0240767 A1
(43) Pub. Date: Aug. 20, 2026

(54) MASSAGING BALM FORMULATION

(71) Applicant: CHATTEM INC., Morristown, NJ (US)

(72) Inventors: David SHARP, Hixon, TN (US); Congling ZHANG, Apison, TN (US)

(21) Appl. No.: 19/489,508

(22) PCT Filed: Jun. 2, 2023
(86) PCT No.: PCT/US2023/067839

§ 371 Date: Dec. 2, 2025

(51) Int. Cl. A61K 9/06; A61K 31/045; A61K 31/125; A61K 47/44; A61K 47/46

(52) U.S. Cl. CPC A61K 9/06; A61K 31/045; A61K 31/125; A61K 47/44; A61K 47/46

(57) ABSTRACT

The disclosure is directed to massaging balm formulations contain a pain or itch relieving active. In certain embodiments, the formulations contain: from about 1% to about 16% (w/w) of menthol; from about 3% to about 11% (w/w) of camphor, from about 62% to about 95.5% (w/w) of a plurality of waxes; from about 0. 1% to about 10% (w/w) of a plasticizer; and from about 0% to 2% (w/w) of essential oils. In other embodiments, the formulations contain: from about 14% to about 18% (w/w) of menthol; from about 8% to about 14% (w/w) of camphor; one or more moisturizer; one or more skin conditioning agents; one or more fragrance; and from about 0.75% to about 2% (w/w) of a plasticizer. These massaging balm formulations may be placed on an applicator. The disclosure also includes using the applicator and/or massaging balm formulation to treat pain and/or relieve itch. When the applicator is used, pain is treated through the massaging action of the applicator and analgesic action of camphor and menthol.

### FIELD OF THE INVENTION

This disclosure is directed to massaging balm formulations containing menthol and camphor as the active ingredient.

### BACKGROUND OF THE INVENTION

Whether it is arthritis pain, sports injury or other pain, topical pain relievers are commonly used treat a variety of pains including arthritis pain and pain from sports injuries.

Topical application of pain relievers commonly involves applying a gel or cream containing the pain reliever to the skin of the patient. However, patients may find it difficult to apply a gel or cream evenly. As gels and creams are typically spread over the area to be treated by hand, patients must deal with the smell of the pain reliever on hands which has to be washed off.

Furthermore, current topical pain relievers on the market provide pain relief either through pain relief actives or massaging applicator rather than combining both means.

Accordingly, what is needed is an improved topical formulation that can easily applied to the skin and can be used as a massaging applicator.

### SUMMARY OF THE INVENTION

The present disclosure is massaging balm formulations. The formulations of the disclosure generally contain: a pain or itch relieving active; one or more moisturizers; one or more skin conditioning agents; one or more plasticizers, and may include one or more fragrance. In one embodiment, the pain or itch relieving active is selected from menthol, camphor, methyl salicylate, lidocaine, capsaicin, hydrocortisone, diphenhydramine, or combinations thereof. In certain embodiments, the pain or itch relieving active is menthol and camphor. In a specific embodiment, menthol and camphor are the only pain or itch relieving active in the formulation. In another specific embodiment, menthol is the only pain or itch relieving active in the formulation.

One embodiment of the invention is a massaging ball formulation containing from about 1% to about 16% (w/w) of menthol; from about 3% to about 11% (w/w) of camphor; one or more moisturizers and skin conditioning agents; from about 0.1% to about 10% (w/w) of a plasticizer; and optionally one or more fragrances. Another embodiment of the invention is a massaging ball formulation containing from about 14% to about 18% (w/w) of menthol; from about 8% to about 14% (w/w) of camphor; one or more moisturizers; one or more skin conditioning agents; from about 0.75% to about 2% (w/w) of a plasticizer; and optionally one or more fragrances. Yet another embodiment of the invention is a massaging ball formulation containing from about 1% to about 16% (w/w) of menthol; from about 3% to about 11% (w/w) of camphor, from about 62% to about 95.5% (w/w) of a plurality of waxes; from about 0.1% to about 10% (w/w) of a plasticizer; from about 0% to 2% (w/w) of essential oils. In certain embodiments, the formulations also contain from about 10% to about 60% (w/w) of methyl salicylate.

In certain embodiment, the plasticizer is a pliable polyester wax such as e.g., a synthetic beeswax. In other embodiments, the plasticizer is selected from the group consisting of C₁₈₋₃₈ alkyl hydroxystearoyl stearate, polyhydroxystearic acid, beeswax, candelilla wax, behenyl behenate, and C₂₀₋₄₀ alkyl stearate.

Various moisturizers can be used in the massaging balm formulations. In one embodiment, the one or more moisturizers is selected from the group consisting of sweet almond oil, sunflower seed wax, carnauba wax, cocoa seed butter, coconut oil, avocado oil or butter, and combinations thereof.

Various fragrances can be used with the formulations. In certain embodiments, the one or more fragrances is selected from the group consisting of eucalyptus oil, grapefruit peel oil, peppermint oil, spearmint oil, and combinations thereof.

One embodiment of the disclosure is a massaging ball formulation containing: from about 14% to about 18% (w/w) of menthol; from about 8% to about 14% (w/w) of camphor; sweet almond (Prunus amygdalus dulcis) oil; sunflower (Helianthus annuus) seed wax; cocoa (Theobroma cacao) seed butter; coconut oil (Cocos nucifera); avocado (Persea gratissima) oil or butter; carnauba (Copemica cerifera) wax; eucalyptus (Eucalyptus globulus) oil; grapefruit (Citrus paradisi) peel oil; peppermint (Mentha piperita) oil; spearmint (Mentha viridis) oil; and from about 0.75% to about 2% (w/w) of a plasticizer.

Another embodiment is a massaging balm formulation containing: from about 14% to about 18% (w/w) of menthol; from about 8% to about 14% (w/w) of camphor; from about 20% to about 30% (w/w) of sweet almond oil; from about 10% to about 20% (w/w) sunflower seed wax; from about 8% to about 16% (w/w) cocoa seed butter; from about 7% to about 15% (w/w) coconut oil; from about 1% to about 6% (w/w) avocado oil or from about 3% to about 10% (w/w) avocado butter; from about 0.5% to about 2.5% (w/w) carnauba wax; from about 0.01% to about 0.5% (w/w) eucalyptus oil; from about 0.01% to about 0.5% (w/w) grapefruit peel oil; from about 0.01% to about 0.5% (w/w) peppermint oil; from about 0.01% to about 0.5% (w/w) spearmint oil; and from about 0.75% to about 2% (w/w) of a plasticizer (such as a pliable polyester wax e.g. synthetic beeswax).

Another embodiment is a massaging balm formulation only containing: from about 14% to about 18% (w/w) of menthol; from about 8% to about 14% (w/w) of camphor; from about 20% to about 30% (w/w) of sweet almond oil; from about 10% to about 20% (w/w) sunflower seed wax; from about 8% to about 16% (w/w) cocoa seed butter; from about 7% to about 15% (w/w) coconut oil; from about 1% to about 6% (w/w) avocado oil or from about 3% to about 10% (w/w) avocado butter; from about 0.5% to about 2.5% (w/w) carnauba wax; from about 0.01% to about 0.5% (w/w) eucalyptus oil; from about 0.01% to about 0.5% (w/w) grapefruit peel oil; from about 0.01% to about 0.5% (w/w) peppermint oil; from about 0.01% to about 0.5% (w/w) spearmint oil; and from about 0.75% to about 2% (w/w) of a plasticizer (such as a pliable polyester wax e.g. synthetic beeswax).

Yet another embodiment, is a kit comprising the massaging balm formulation and an applicator. In a certain embodiment, the applicator is approximately circular and wherein the applicator comprises a grid on which the formulation is placed.

In certain aspects of the disclosure these massaging balm formulation are placed on an applicator, such e.g., a grid, to provide a massaging function as well as melting/dispensing of the pain or itch relieving active, such as e.g., menthol and camphor.

Other features and advantages of the invention will be apparent from the detailed description and the examples that follow.

### BRIEF DESCRIPTION OF THE DRAWINGS

The foregoing summary, as well as the following detailed description of the invention, will be better understood when read in conjunction with the appended figure. For the purpose of illustrating the invention, the figure demonstrates embodiments of the present invention. It should be understood, however, that the invention is not limited to the precise arrangements, examples, and instrumentalities shown.

FIG. 1 shows the effects of various amounts of plasticizer on formulations containing pain or itch relieving actives (menthol and camphor), moisturizers, skin conditioning agents, and fragrances.

FIG. 2A shows a cross-section view of a formulation containing menthol, camphor, moisturizers, skin conditioning agents, and fragrances. FIG. 2B shows a cross-section view of a formulation containing pain or itch relieving actives (menthol and camphor), moisturizers, skin conditioning agents, fragrances, and 1% of plasticizer.

FIG. 3 shows average forces necessary for various penetration depth comparing Formula A (pain or itch relieving actives (menthol and camphor), moisturizers, skin conditioning agents, and fragrances) and Formula B (pain or itch relieving actives (menthol and camphor), moisturizers, skin conditioning agents, fragrances, and 1% of plasticizer).

### DETAILED DESCRIPTION

This disclosure is directed massaging balm formulations suitable for topical application. The massaging balm formulations contain a pain or itch relieving active. In one embodiment, the pain or itch relieving active is selected from menthol, camphor, methyl salicylate, lidocaine, capsaicin, hydrocortisone, diphenhydramine, or combinations thereof. In certain embodiments, the pain or itch relieving active is menthol and camphor. The formulations are designed to provide a massaging function as well as dispersing pain or itch relieving actives (such as e.g., menthol and camphor) at maximum strength (e.g., per OTC monograph) to the treated area during application. The formulations also designed to have a non-greasy and moisturizing after feel. Addition of a plasticizer such as, e.g., synthetic beeswax, ensures the uniformity of the ball during manufacturing process and consistent consumer experience throughout the life span of this product.

Without being bound by theory, the formulations have optimized rigidity and structural integrity of the hot fill solid to provide a massage function while melting the pain or itch relieving active ingredients (e.g., menthol and camphor). Addition of plasticizer provides homogeneous structure and prevents migration of the APIs.

The formulations of the disclosure are uniquely formulated to provide improved adhesion to an applicator, such as an applicator grip. Accordingly, the formulations are suitable for use as massaging applicator. Furthermore, the formulations of the disclosure maintain their shape and are spreadable.

The disclosure is also related to the formulation on a mess free applicator. When used on such an applicator, pain or itch relieving active in the formulation can be massaged into the pain area to provide pain and/or itch relief. Thus, the formulations of an applicator provide pain relief through pain relief actives (such as e.g., menthol and camphor) and a massaging functionality.

For clarity of disclosure, and not by way of limitation, the detailed description of the invention is divided into subsections that describe or illustrate certain features, embodiments, or applications of the present invention.

### Definitions

As used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the singular forms “a,” “an,” and “the” include the plural reference. Reference to a particular numerical value includes at least that particular value unless the context clearly indicates otherwise. Thus, for example, reference to “a substance” is a reference to at least one of such substances and equivalents thereof known to those skilled in the art, and so forth.

When a value is expressed as an approximation by use of “about,” it will be understood that the particular value forms another embodiment. In general, use of “about” indicates approximations that can vary depending on the desired properties sought to be obtained by the disclosed subject matter and is to be interpreted in the specific context in which it is used. The person skilled in the art will be able to interpret this as a matter of routine. In some instances, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

As used herein, while data is given with full disclosure of all significant figures, those of skilled in the art would understand that the data can also be understood when rounded to 1 or 2 decimal places.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself. Before certain embodiments are described in greater detail, it is to be understood that this invention is not limited to certain embodiments described, and as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

The terms “patient” or “subject” as used herein are interchangeable and refer to a mammalian animal. In some embodiments, the patient or subject is a human. In some embodiments, the patient is an adult, i.e., 18 years of age or older. In other embodiments, the patient is a child, i.e., under the age of 18.

The term “treating” includes ameliorating a disease or disorder (i.e., arresting or reducing the development of the disease or at least one of the clinical symptoms thereof). In some embodiments, “treating” refers to ameliorating at least one physical parameter, which may not be discernible by the subject. In other embodiments, “treating” refers to modulating the disease or disorder, either physically (e.g., stabilizing a discernible symptom), physiologically (e.g., stabilizing a physical parameter), or both. In further embodiments, “treating” refers to delaying the onset of the disease or disorder.

### Massaging Balm Formulations

It is an object of the present invention to provide massaging balm formulations suitable for topical application that contain pain or itch relieving active(s). In one embodiment, the pain or itch relieving active is selected from menthol, camphor, methyl salicylate, lidocaine, capsaicin, cortisone, or combinations thereof. In certain embodiments, the pain or itch relieving active is menthol and camphor. In other embodiments, menthol is the only pain or itch relieving active in the formulation. The formulations can include: the pain or itch relieving active (such as e.g., external analgesics (menthol and camphor); one or more moisturizer; one or more skin conditioning agents; one or more plasticizer, and one or more fragrance.

In some embodiments, the formulations of the invention only contain menthol and camphor as the active pharmaceutical ingredient; they do not contain further active pharmaceutical ingredients.

The formulations of the disclosure are formulated to secure the formulation on an applicator. In certain embodiments, they are also formulated so that they can be used to provide a massaging function when used to treat pain and/or relieve itch.

In one embodiment, the massaging ball formulation contains from about 1% to about 16% (w/w) of menthol; from about 3% to about 11% (w/w) of camphor; one or more moisturizers; one or more skin conditioning agents; from about 0.1% to about 10% (w/w) of a plasticizer; and optionally one or more fragrances. In an alternate embodiment, the massaging balm formulation contains: from about 14% to about 18% (w/w) of menthol; from about 8% to about 14% (w/w) of camphor; one or more moisturizer; one or more skin conditioning agents; one or more fragrance; and from about 0.75% to about 2% (w/w) of a plasticizer. In another embodiment, the massaging ball formulation contains: from about 1% to about 16% (w/w) of menthol; from about 3% to about 11% (w/w) of camphor, from about 62% to about 95.5% (w/w) of a plurality of waxes; from about 0.1% to about 10% (w/w) of a plasticizer; from about 0% to 2% (w/w) of essential oils. In an alternate embodiment, the massaging ball formulation contains from about 1% to about 16% (w/w) of menthol; from about 3% to about 11% (w/w) of camphor; from about 10-60% (w/w) of methyl salicylate; one or more moisturizers; one or more skin conditioning agents; from about 0.1% to about 10% (w/w) of a plasticizer; and optionally one or more fragrances.

In another embodiment, the massaging balm formulation contains: from about 14% to about 18% (w/w) of menthol; from about 8% to about 14% (w/w) of camphor; sweet almond (Prunus amygdalus dulcis) oil; sunflower (Helianthus annuus) seed wax; cocoa (Theobroma cacao) seed butter; coconut oil (Cocos nucifera); avocado (Persea gratissima) oil or butter; carnauba (Copemica cerifera) wax; eucalyptus (Eucalyptus globulus) oil; grapefruit (Citrus paradisi) peel oil; peppermint (Mentha piperita) oil; spearmint (Mentha viridis) oil; and from about 0.75% to about 2% (w/w) of a plasticizer.

### External Analgesics Menthol and Camphor

In certain embodiments, menthol is present in an amount from about 1% to about 16% (w/w), alternatively from about 1% to about 18% (w/w), alternatively from about 14% to about 18% (w/w), alternatively from about 15% to about 17% (w/w), alternatively from about 15.5% to about 16.5% (w/w).

In other embodiments, camphor is present in an amount from about 3% to about 11% (w/w), from about 3% to about 14% (w/w), from about 8% to about 14% (w/w), alternatively from about 9% to about 13% (w/w), alternatively from about 9.5% to about 12% (w/w).

In certain embodiments, menthol is present in an amount from about 1% to about 16% (w/w), alternatively from about 1% to about 18% (w/w), alternatively from about 14% to about 18% (w/w), alternatively from about 15% to about 17% (w/w), alternatively from about 15.5% to about 16.5% and camphor is present in an amount from about 14% to about 18% (w/w), alternatively from about 15% to about 17% (w/w), alternatively from about 15.5% to about 16.5% (w/w).

In other embodiments, the massaging balm formulation contains: a pain or itch relieving active selected from the group consisting of menthol, camphor, methyl salicylate, lidocaine, capsaicin, diphenhydramine, hydrocortisone, or combinations thereof; from about 0.1% to about 10% of a plasticizer; and an essential oil. In certain embodiments, the essential oil is lavender oil, rose oil, eucalyptus (Eucalyptus globulus) oil, a citrus oil (such as e.g., grapefruit (Citrus paradisi) peel oil), peppermint (Mentha piperita) oil, spearmint (Mentha viridis) oil, cinnamon oil, or combination thereof. In one specific embodiment the pain or itch relieving active is menthol and the essential oil is lavender oil.

### One or More Moisturizers or Skin Conditioning Agents

In one embodiment, the one or more moisturizers or skin conditioning agents is selected from the group consisting of sweet almond (Prunus amygdalus dulcis) oil, sunflower (Helianthus annuus) seed wax, cocoa (Theobroma cacao) seed butter, coconut oil (Cocos nucifera), avocado (Persea gratissima) oil or butter, carnauba (Copemica cerifera) wax and combinations thereof. In certain embodiments, the one or more moisturizers is each of sweet almond (Prunus amygdalus dulcis) oil, sunflower (Helianthus annuus) seed wax, cocoa (Theobroma cacao) seed butter, carnauba (Copemica cerifera) wax. coconut oil (Cocos nucifera), avocado (Persea gratissima) oil or butter. In some embodiments the avocado (Persea gratissima) butter is a mixture of avocado (Persea gratissima) oil and a hydrogenated vegetable oil. In certain embodiments, avocado (Persea gratissima) butter is a mixture of about 60% w/w of avocado (Persea gratissima) oil and about 40% w/w of a hydrogenated vegetable oil. In certain embodiments, the formulations contain from about 50% to about 80% (w/w), alternatively from about 60% to about 80% (w/w), alternatively from about 65% to about 75% (w/w) of the moisturizer.

In some embodiments, the sunflower (Helianthus annuus) seed wax is present in an amount from about 10% to about 20% (w/w), alternatively from about 12% to about 16% (w/w), alternatively from about 13% to about 15% (w/w). In other embodiments, the cocoa (Theobroma cacao) seed butter is present in an amount from about 8% to about 16% (w/w), alternatively from about 9% to about 15% (w/w), alternatively from about 10% to about 14% (w/w). In alternate embodiments, the coconut oil (Cocos nucifera) is present in an amount from about 7% to about 15% (w/w), alternatively from about 8% to about 14% (w/w), alternatively from about 10% to about 13% (w/w). In further embodiments, the avocado (Persea gratissima) oil is present in an amount from about 1% to about 6% (w/w), alternatively from about 1% to about 5% (w/w), alternatively from about 2% to about 4% (w/w). In additional embodiments, the avocado (Persea gratissima) butter is present in an amount from about 3% to about 10% (w/w), alternatively from about 4% to about 8% (w/w), alternatively from about 4% to about 7% (w/w).

In certain embodiments, the carnauba (Copemica cerifera) wax is present in an amount from about 0.5% to about 2.5% (w/w), alternatively from about 0.5% to about 2.0% (w/w), alternatively from about 0.8% to about 1.5% (w/w).

In certain embodiments, the one or more moisturizers and skin conditioning agents are each of sweet almond (Prunus amygdalus dulcis) oil, sunflower (Helianthus annuus) seed wax, cocoa (Theobroma cacao) seed butter, carnauba (Copemica cerifera) wax, coconut oil (Cocos nucifera), avocado (Persea gratissima) oil or butter present in the amounts listed in Tables A and B below.

TABLE A

| Moisturizer | Amount (w/w) | | |
|---|---|---|---|
| sweet almond (Prunus | from about | from about | from about 24% to |
| Amygdalus Dulcis) oil | 20% to | 22% to | about 27% |
| | about 30% | about 28% | |
| sunflower (helianthus | from about | from about | from about 13% to |
| annuus) seed wax | 10% to | 12% to | about 15% |
| | about 20% | about 16% | |
| cocoa (Theobroma | from about | from about | from about 5% to |
| cacao) seed butter | 0.1% to | 1% to | about 20% o |
| | about 30% | about 25% | |
| | of each | of each | |
| coconut oil (Cocos | from about | from about | from about 5% to |
| nucifera) | 0.1% to | 1% to | about 20% o |
| | about 30% | about 25% | |
| | of each | of each | |
| avocado (Persea | from about | from about | from about 5% to |
| gratissima) oil | 0.1% to | 1% to | about 20% o |
| | about 30% | about 25% | |
| | of each | of each | |
| avocado (Persea | from about | from about | from about 5% to |
| gratissima) butter | 0.1% to | 1% to | about 20% o |
| | about 30% | about 25% | |
| | of each | of each | |
| carnauba (Copemica | from about | from about | from about 5% to |
| cerifera) wax | 0.1% to | 1% to | about 20% o |
| | about 30% | about 25% | |
| | of each | of each | |

TABLE B

| Moisturizer | Amount (w/w) | | |
|---|---|---|---|
| sweet almond (Prunus | from about 20% | from about 22% | from about 24% to |
| Amygdalus Dulcis) oil | to about 30% | to about 28% | about 27% |
| sunflower (helianthus | from about 10% | from about 12% | from about 13% to |
| annuus) seed wax | to about 20% | to about 16% | about 15% |
| cocoa (Theobroma cacao) | from about 8% to | from about 9% to | from about 10% to |
| seed butter | about 16% | about 15% | about 14% |
| coconut oil (Cocos | from about 7% to | from about 8% to | from about 10% to |
| nucifera) | about 15% | about 14% | about 13% |
| avocado (Persea | from about 1% to | from about 1% to | from about 2% to |
| gratissima) oil | about 6% | about 5% | about 4% |
| avocado (Persea | from about 3% to | from about 4% to | from about 4% to |
| gratissima) butter | about 10% | about 8% | about 7% |
| carnauba (Copemica | from about 0.5% | from about 0.5% | from about 0.8% to |
| cerifera) wax | to about 2.5% | to about 2% | about 1.5% |

In the Tables above, the various amounts for each moisturizer can be used in combination with each other. For example, any of the amounts of sunflower seed wax may be used with any of the amounts of sweet almond oil, etc.

In further embodiments, the one or more moisturizers and skin conditioning agents are selected from the group consisting of Castor Wax, candelilla wax, jojoba wax, jojoba oil, jojoba esters, beeswax, orange wax, sunflower wax, soy wax, rice bran wax, carnauba wax, Stearic Acid, Stearic Alcohol, Ceteryl Alcohol, Cetyl Alcohol, Rapeseed Oil, Behenyl Alcohol, Behentrimonium Methosulfate, Behenyl Behenate, Polyhydroxystearic Acid, Polyglyceryl-10 Stearate, Euphorbia cerifera (Candelilla) Wax, Sodium Stearoyl Lactylate, Polyglyceryl-3 Stearate, Sodium Stearoyl Lactylate, Polyglyceryl-3 Esters, and combination thereof.

The cocoa (Theobroma cacao) seed butter can be deodorized.

### One or More Plasticizers

In other embodiments, the one or more plasticizers are a pliable polyester wax. In certain embodiments, the plasticizer is a synthetic beeswax, such as e.g., Kester Wax K-82P. In other embodiments, the plasticizer is selected from the group consisting of C₁₈₋₃₈ alkyl hydroxystearoyl stearate, polyhydroxystearic acid, beeswax, candelilla wax, behenyl behenate, and C₂₀₋₄₀ alkyl stearate. The one or more plasticizers, such as e.g., a pliable polyester wax or a synthetic beeswax, can be present from about 0.1% to about 10% (w/w), alternatively from about 0.2% to about 8% (w/w), alternatively from about 1% to about 9% (w/w), alternatively from about 2% to about 7%, alternatively from about 0.75% to about 2% (w/w), alternatively from about 0.8% to about 1.8% (w/w), alternatively from about 0.8% to about 1.4% (w/w), alternatively at least 0.7% (w/w), alternatively at least 0.8% (w/w), alternatively at least 0.9% (w/w).

### One or More Fragrances

In additional embodiments, the one or more fragrances is selected from the group consisting of eucalyptus (Eucalyptus globulus) oil, grapefruit (Citrus paradisi) peel oil, peppermint (Mentha piperita) oil, spearmint (Mentha viridis) oil, and combinations thereof. In other embodiments, the one or more fragrances is eucalyptus (Eucalyptus globulus) oil, grapefruit (Citrus paradisi) peel oil, peppermint (Mentha piperita) oil, and spearmint (Mentha viridis) oil.

In certain embodiments, the fragrance is present in a total amount from about 0.01% to about 1% (w/w), alternatively from about 0.01% to about 0.8% (w/w), alternatively from about 0.01% to about 0.7% (w/w), alternatively from about 0.04% to about 2% (w/w), alternatively from about 0.2% to about 1.2% (w/w), alternatively from about 0.2% to about 0.8% (w/w). In an embodiment, the eucalyptus (Eucalyptus globulus) oil is present in an amount from about 0.01% to about 1% (w/w), alternatively from about 0.01% to about 0.8% (w/w), alternatively from about 0.01% to about 0.7%, alternatively from about 0.01% to about 0.5% (w/w), alternatively from about 0.05% to about 0.3% (w/w), alternatively from about 0.05% to about 0.2% (w/w). In another embodiment, the grapefruit (Citrus paradisi) peel oil is present in an amount from about 0.01% to about 1% (w/w), alternatively from about 0.01% to about 0.8% (w/w), alternatively from about 0.01% to about 0.7% (w/w), alternatively from about 0.01% to about 0.5% (w/w), alternatively from about 0.05% to about 0.3% (w/w), alternatively from about 0.05% to about 0.2% (w/w). In yet another embodiment, the peppermint (Mentha piperita) oil is present in an amount from about 0.01% to about 1% (w/w), alternatively from about 0.01% to about 0.8% (w/w), alternatively from about 0.01% to about 0.7%, alternatively from about 0.01% to about 0.5% (w/w), alternatively from about 0.05% to about 0.3% (w/w), alternatively from about 0.05% to about 0.2% (w/w). In yet another embodiment, the spearmint (Mentha viridis) oil is present in an amount from about 0.01% to about 1% (w/w), alternatively from about 0.01% to about 0.8% (w/w), alternatively from about 0.01% to about 0.7%, alternatively from about 0.01% to about 0.5% (w/w), alternatively from about 0.05% to about 0.3% (w/w), alternatively from about 0.05% to about 0.2% (w/w). In other embodiments, the one or more fragrances is eucalyptus (Eucalyptus globulus) oil, grapefruit (Citrus paradisi) peel oil, peppermint (Mentha piperita) oil, and spearmint (Mentha viridis) oil and each of which are present in an amount from about 0.01% to about 1% (w/w), alternatively from about 0.01% to about 0.8% (w/w), alternatively from about 0.01% to about 0.7%, alternatively from about 0.01% to about 0.5% (w/w), alternatively from about 0.05% to about 0.3% (w/w), alternatively from about 0.05% to about 0.2% (w/w).

Tables 1 and 2 show exemplary massaging balm formulations according to certain embodiments of the invention. The various amounts for components in each of the embodiments of Tables 1 and Tables 2 may be combined. In addition, in some embodiments the exemplary balm formulations may contain an additional pain or itch relieving active. The additional pain or itch relieving active is selected from methyl salicylate, lidocaine, capsaicin, diphenhydramine, hydrocortisone, or combinations thereof.

TABLE 1

| | Embodiment | | | |
|---|---|---|---|---|
| | 1-1 | 1-2 | 1-3 | 1-4 |
| | Amount | Amount | Amount | Amount |
| Component | (w/w) | (w/w) | (w/w) | (w/w) |
| menthol | from about 14% | from about 15% | from about | from about 1% |
| | to about 18% | to about 17% | 15.5% to about | to about 16% |
| | | | 16.5% | |
| camphor | from about 8% | from about 9% | from about | from about 3% |
| | to about 14% | to about 13% | 9.5% to about | to about 11% |
| | | | 12% | |
| sweet almond | from about 20% | from about 22% | from about 24% | from about |
| (Prunus Amygdalus | to about 30% | to about 28% | to about 27% | 0.1% to about |
| Dulcis) oil | | | | 30% |
| sunflower | from about 10% | from about 12% | from about 13% | from about |
| (helianthus annuus) | to about 20% | to about 16% | to about 15% | 0.1% to about |
| seed wax | | | | 30% |
| cocoa (Theobroma | from about 8% | from about 9% | from about 10% | from about |
| cacao) seed butter | to about 16% | to about 15% | to about 14% | 0.1% to about |
| | | | | 30% |
| coconut oil (Cocos | from about 7% | from about 8% | from about 10% | from about |
| nucifera) | to about 15% | to about 14% | to about 13% | 0.1% to about |
| | | | | 30% |
| avocado (Persea | from about 1% | from about 1% | from about 2% | from about |
| gratissima) oil | to about 6% | to about 5% | to about 4% | 0.1% to about |
| | | | | 30% |
| carnauba (Copemica | from about | from about | from about | from about |
| cerifera) wax | 0.5% to about | 0.5% to about | 0.8% to about | 0.1% to about |
| | 2.5% | 2.0% | 1.5% | 30% |
| plasticizer (pliable | from about | from about | from about | from about |
| polyester wax, e.g., | 0.75% to about | 0.8% to about | 0.8% to about | 0.1% to about |
| synthetic beeswax) | 2% | 1.8% | 1.4% | 10% |
| eucalyptus | from about | from about | from about | from about |
| (Eucalyptus globulus) | 0.01% to about | 0.05% to about | 0.05% to about | 0.01% to about |
| oil | 0.5% | 0.3% | 0.2% | 2% |
| grapefruit (Citrus | from about | from about | from about | from about |
| paradisi) pccl oil | 0.01% to about | 0.05% to about | 0.05% to about | 0.01% to about |
| | 0.5% | 0.3% | 0.2% | 2% |
| peppermint (Mentha | from about | from about | from about | from about |
| piperita) oil | 0.01% to about | 0.05% to about | 0.05% to about | 0.01% to about |
| | 0.5% | 0.3% | 0.2% | 2% |
| spearmint (Mentha | from about | from about | from about | from about |
| viridis) oil | 0.01% to about | 0.05% to about | 0.05% to about | 0.01% to about |
| | 0.5% | 0.3% | 0.2% | 1% |

TABLE 2

| | Embodiment | | | |
|---|---|---|---|---|
| | 2-1 | 2-2 | 2-3 | 2-4 |
| | Amount | Amount | Amount | Amount |
| Component | (w/w) | (w/w) | (w/w) | (w/w) |
| menthol | from about 14% | from about 15% | from about | from about 1% |
| | to about 18% | to about 17% | 15.5% to about | to about 16% |
| | | | 16.5% | |
| camphor | from about 8% | from about 9% | from about | from about 3% |
| | to about 14% | to about 13% | 9.5% to about | to about 11% |
| | | | 12% | |
| sweet almond (Prunus | from about 20% | from about 22% | from about 24% | from about |
| Amygdalus Dulcis) oil | to about 30% | to about 28% | to about 27% | 0.1% to about |
| | | | | 30% |
| sunflower (helianthus | from about 10% | from about 12% | from about 13% | from about |
| annuus) seed wax | to about 20% | to about 16% | to about 15% | 0.1% to about |
| | | | | 30% |
| cocoa (Theobroma | from about 8% | from about 9% | from about 10% | from about |
| cacao) seed butter | to about 16% | to about 15% | to about 14% | 0.1% to about |
| | | | | 30% |
| coconut oil (Cocos | from about 7% | from about 8% | from about 10% | from about |
| nucifera) | to about 15% | to about 14% | to about 13% | 0.1% to about |
| | | | | 30% |
| avocado (Persea | from about 3% | from about 4% | from about 4% | from about |
| gratissima) butter | to about 10% | to about 8% | to about 7% | 0.1% to about |
| | | | | 30% |
| carnauba (Copemica | from about | from about | from about | from about |
| cerifera) wax | 0.5% to about | 0.5% to about | 0.8% to about | 0.1% to about |
| | 2.5% | 2.0% | 1.5% | 30% |
| plasticizer (pliable | from about | from about | from about | from about |
| polyester wax, e.g., | 0.75% to about | 0.8% to about | 0.8% to about | 0.1% to about |
| synthetic beeswax) | 2% | 1.8% | 1.4% | 10% |
| eucalyptus | from about | from about | from about | from about |
| (Eucalyptus globulus) | 0.01% to about | 0.05% to about | 0.05% to about | 0.01% to about |
| oil | 0.5% | 0.3% | 0.2% | 2% |
| grapefruit (Citrus | from about | from about | from about | from about |
| paradisi) peel oil | 0.01% to about | 0.05% to about | 0.05% to about | 0.01% to about |
| | 0.5% | 0.3% | 0.2% | 2% |
| peppermint (Mentha | from about | from about | from about | from about |
| piperita) oil | 0.01% to about | 0.05% to about | 0.05% to about | 0.01% to about |
| | 0.5% | 0.3% | 0.2% | 2% |
| spearmint (Mentha | from about | from about | from about | from about |
| viridis) oil | 0.01% to about | 0.05% to about | 0.05% to about | 0.01% to about |
| | 0.5% | 0.3% | 0.2% | 1% |

For example, any amount of menthol and/or camphor listed in Table 1 and Table 2 may be combined with any of the amounts of sweet almond (Prunus Amygdalus Dulcis) oil; sunflower (Helianthus annuus) seed wax; cocoa (Theobroma cacao) seed butter; coconut oil (Cocos nucifera); avocado (Persea gratissima) oil or butter; carnauba (Copemica cerifera) wax; eucalyptus (Eucalyptus globulus) oil; grapefruit (Citrus paradisi) peel oil; peppermint (Mentha piperita) oil; spearmint (Mentha viridis) oil; and/or plasticizer listed in Table 1 and Table 2.

### Kits

In another embodiment, the invention is directed to a kit containing a massaging balm formulation as disclosed herein. The kit may optionally include an applicator and/or instructions for use.

### Applicators

In one embodiment of the disclosure, the massaging balm formulation is on a hand-held applicator. In certain embodiments, the massaging balm formulation is placed on a hand-held applicant in an approximately semi-spherical form.

The hand-held applicator may include a semi-circular substrate on which the massaging balm formulation is placed. In one embodiment, the substrate may comprise a grid. In another embodiment, the substrate may comprise an approximately shaped convex grid.

The hand-held applicator may have two sides. One side of the hand-held applicator is configured to accept the massaging balm formulation, the other side is configured for gripping. In certain embodiments, the hand-held applicator may also include a cover to protect the massaging balm formulation on the hand-held applicator when not in use.

In some embodiments, the side of the applicator on which the massaging balm formulation is placed is dome shaped.

In certain embodiments, the hand-held applicator is an applicator as disclosed in PCT Application No. ______, filed Jun. 2, 2023, entitled “Massaging Balm Applicator” (Attorney Docket No. 070589.12791 (188WO1)), the disclosure of which is incorporated in its entirety as it pertains to applicators.

One embodiment of the disclosure is directed to an applicator comprising a formulation of the disclosure. The applicator has a surface in contact with the formulation, i.e., on which the formulation is paced.

In other embodiments, the applicator is hand-held. In certain embodiments, the applicator is approximately circular and wherein the applicator comprises a grid in contact with the formulation.

The grip may be concave (i.e., dome-shaped). In certain embodiments, when the formulation is on the applicator, the formulation on the applicator is shaped as a semi-sphere (hemispherical). The applicator may also include a cover. The cover may be shaped a semi-sphere (hemispherical). In other embodiments, the cover may be shaped like a petal. In certain embodiments, the applicator maybe circular or rectangular.

### Methods of Placing Formulations on Applicators

Yet another aspect of the disclosure is directed to methods of placing the formulations described herein on applicators, such as the applicators described above.

The methods generally involve placing/contacting the formulations of the disclosure with an applicator (substrate) and, if desired, forming the formulation into a desired shape.

Accordingly, one embodiment of the disclosure is a method of generating an applicator comprising a formulation disclosed herein. The method includes the steps of providing an applicator having a top and bottom surface; and contacting the formulation with the top surface of the applicator. The bottom surface of the applicator is configured for hand-holding of the applicator.

In certain embodiments, the step of contacting includes hot melting the formulation on the top surface of the applicator. In other embodiments, the step of contacting includes hot melting the formulation into a forming cup.

The method can also include further steps. In one embodiment, the method includes generating the formulation prior to contacting. In another embodiment, the method include shaping the formulation into a desired shape. In one embodiment, the method includes shaping the formulation into approximately a semi-sphere.

In certain embodiments, the shaping includes use of a forming cup to shape the formulation into a desired shape. The desired shape can be hemispherical. In certain embodiments in which the forming cup is used, the method includes use of a mold release agent to release the formulation from the forming cup.

In certain embodiments of the disclosure, the applicator includes a grip.

In some embodiments, the top surface of the applicator is concave (i.e., domed).

Uses of the Formulations and Uses of Applicators with the Formulations

The massaging balm formulations described herein are useful to reduce or ameliorate (i.e., relief) pain and/or relieve itch in a patient in need thereof. The methods include topically applying the formulation to the skin of a patient.

In one embodiment of the disclosure, the massaging balm formulation is placed on a hand-held applicator. In certain embodiments, the massaging balm formulation is placed on a hand-held applicator in an approximately semi-spherical form. Accordingly, the method includes rubbing the skin of a patient with a hand-held applicator comprising the formulations of the disclosure to treat pain via both pain relief actives and massage.

One embodiment of the disclosure is use of the formulations of the disclosure to treat pain. Another embodiment of the disclosure is use of an applicator comprising the formulations of the disclosure to treat pain. Another embodiment of the disclosure is use of the formulations of the disclosure to treat pain and/or relieve itch. Yet another embodiment of the disclosure is use of an applicator comprising the formulations of the disclosure to treat pain and/or relieve itch.

Yet another embodiment is use of the formulations of the disclosure in the manufacture of a medicament to treat pain. Yet an alternate embodiment is use of an applicator comprising the formulations of the disclosure to treat pain. Yet a further embodiment is use of the formulations of the disclosure in the manufacture of a medicament to treat pain and/or relieve itch. Yet an alternate embodiment is use of an applicator comprising the formulations of the disclosure to treat pain and/or relieve itch.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### EXAMPLES

### Example 1—Impact of Amount of Plasticizer for Generating Lotion Ball Formulations

The suitability of the claimed formulations to form a semi-spherical lotion ball was assessed. For this assessment, formulations having the components shown below in Table 1-1 was tested with varying amounts of a plasticizer (synthetic beeswax Kester Wax K-82P).

Specifically, the formulation was tested using various amounts of plasticizer, ranging from 0.1 to 5%. (w/w). The control contained no plasticizer. Visual observations were conducted.

TABLE 1-1
Components of Tested Formulation

menthol
camphor
sweet almond (Prunus Amygdalus Dulcis) oil
sunflower (helianthus annuus) seed wax
cocoa (Theobroma cacao) seed butter;
coconut oil (Cocos nucifera)
avocado (Persea gratissima) oil or butter
carnauba (Copemica cerifera) wax
eucalyptus (Eucalyptus globulus) oil
grapefruit (Citrus paradisi) peel oil
peppermint (Mentha piperita) oil
spearmint (Mentha viridis) oil

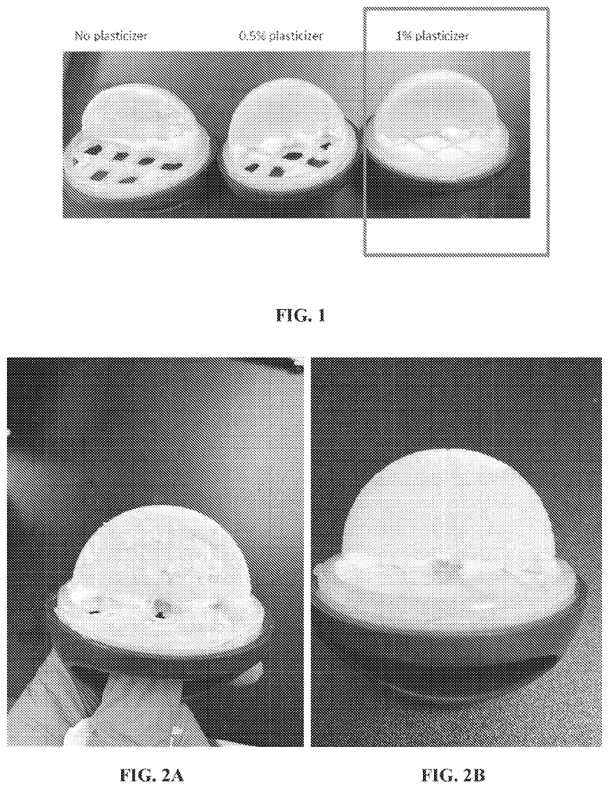

As is evident from the visual observations shown in FIG. 1, the formulations containing 1.0% of the plasticizer showed the best heterogeneity. No difference was observed for amounts >2% of the plasticizer (data not shown).

### Example 2: Evaluation of Product Consistency

Based on the preliminary studies in Example 1, more detailed studies were conducted to investigate the effects of using 1% of a plasticizer (synthetic beeswax Kester Wax K-82P) in formulations containing the components shown in Table 1-1 above.

Specifically, two formulations having the features shown in Table 1-1 were evaluated. The formulations differed from each other.

The first formulation did not contain any plasticizer. The second formulation contained 1% of the plasticizer.

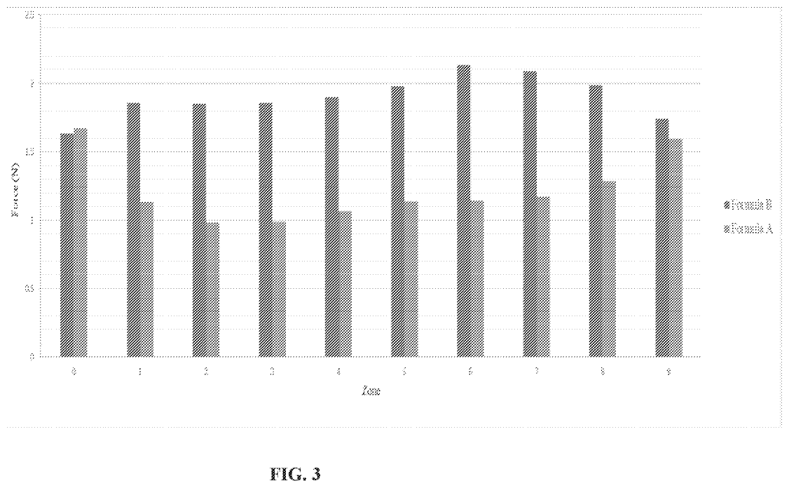

The formulations when placed on grip have a hemispherical shape (see FIG. 2A and FIG. 2B). The formulation lacking the plasticizer showed visible structural heterogeneity when cooled under commercial scale parameters (see FIG. 2A).

When cooled under the same conditions, the formulation containing the plasticizer showed improved structural homogeneity (see FIG. 2B). Further comparison through penetration testing was conducted to assess extent variability within and between formulations.

### Methods

The study was performed by conducting a penetration test using a TA. XTplusC Texture with a TA-052 probe. TA-052 is a stainless-steel punch probe having a diameter of 2 mm and a length of 25 mm.

The study aimed to evaluate the product consistency of two different formulations: Formula A containing the components shown in Table 1-1 above without plasticizer; and Formula B containing the components show in Table 1-1 and 1% of the plasticizer. Five replicates of each formula were used for the studies in this Example.

For the studies, the lotion ball was clamped tightly under the texture analyzer probe using a 3D printed lotion ball holder. A height calibration was performed before each run to determine the top of the lotion ball sample. The probe was allowed to travel at a pre-test velocity of 0.5 mm/sec perpendicular to the direction to the resting surface of the lotion ball.

Once the probe touched the top of the sample, the force required by the probe to maintain a velocity of 0.5 mm/sec penetrating through the lotion ball was recorded. The force required is a representation of the resistance provided by the sample to the penetrating probe and a measure of the structural consistency.

The penetration path length for the probe was kept constant at 24.95 mm (probe length is 25 mm) to avoid any impact of the probe extensions with the lotion ball at the top, and to avoid the tip of the probe hitting the base packaging. The force required by the probe to travel at 0.5 mm/sec was recorded at an average of 2.5 μm intervals. The texture analyzer reached the constant velocity of 0.5 mm/sec within 0.10 sec of penetrating the lotion ball.

All data prior to reaching to the 0.50 mm/sec velocity was discarded. Average data points were generated using the Texture Analyzer software-Exponent Connect of each lot for data analysis.

The average penetration force recorded, was higher for Formula B compared to Formula A. The results of this testing a summarized in Table 2-1 below.

TABLE 2-1
Summary of Testing Data

| | Formula A | Formula B |
|---|---|---|
| Mean Force to travel 24.95 mm (N) | 1.2 | 1.9 |
| Standard Deviation | 0.23 | 0.16 |
| Relative Standard Deviation (%) | 19.2 | 8.2 |
| Median Force (N) | 1.4 | 1.9 |
| Maximum Force (N) | 2.0 | 2.2 |
| Minimum Force (N) | 0.97 | 1.04 |

Specifically, Table 2-1 depicts the data summary of the average force required by the probe to travel 24.95 mm inside the lotion ball. The mean force required by the probe to penetrate Formula B was 1.6 times higher compared Formula A indicating the overall hardness of Formula B to be higher. The percentage deviation of Formula A was 19.2% compared to 8.2% for Formula B.

The percentage deviation is a measure of how the penetration force varied compared to the mean force during the probe travel across the path length of 24.95 mm of the sample. The higher percentage deviation may indicate more inconsistency or lack of uniformity across the sample.

For visualization purposes the penetration distance was divided into ten zones (Zone 0-Zone 9), where Zone 0 represents a penetration depth of 2.5 mm from the top of the lotion ball and Zone 1 represents the next 2.5 mm, that is, penetration depth from 2.5 mm to 5.0 mm. Zone 2-8 were all divided in the subsequent 2.5 mm distances. Zone 9 had a depth of 2.495 mm.

FIG. 3 shows the average forces for each formula at the individual zones. Formula A showed a drop of 32% in the mean force values from Zone 0 to Zone 1, while Formula B showed 13.5% increase for the same Zones. Hence the deviation from the peripheral zone (Zone 0) to the next zone (Zone 1) was significantly higher for Formula A, further indicating lack of uniformity and consistency compared to Formula B.

### CONCLUSION

The penetration studies performed on the provided samples of the two formulas indicated product B to have more product consistency across the path-length of the study compared to Formula A, as shown by the % deviation during the performed studies. An improvement in physical consistency was achieved for Formula B compared to Formula A.

### EMBODIMENTS

Provided here are number embodiments of the disclosed technology. These embodiments are illustrative only and do not limit the scope of the present disclosure or of the claims attached.
Embodiment 1. A massaging balm formulation comprising: a pain or itch relieving active selected from the group consisting of menthol, camphor, methyl salicylate, lidocaine, capsaicin, diphenhydramine, hydrocortisone, or combinations thereof; and from about 0.1% to about 10% of a plasticizer.
Embodiment 2. The massaging balm formulation of embodiment further comprising an essential oil.
Embodiment 3. The massaging balm formulation of embodiment 2, wherein the essential oil is lavender oil.
Embodiment 4. The massaging balm formulation of any one of embodiments 1-3, wherein the pain or itch relieving active is menthol and camphor.
Embodiment 5. The massaging balm formulation of any one of embodiments 1-3, wherein the pain or itch relieving active is menthol.
Embodiment 6. The massaging balm formulation of embodiment 1 wherein the formulation comprises about 1% to about 16% (w/w) of menthol; from about 3% to about 11% (w/w) of camphor, from about 62% to about 95.5% (w/w) of a plurality of waxes; from about 0.1% to about 10% (w/w) of a plasticizer; from about 0% to 2% (w/w) of essential oils.
Embodiment 7. The massaging balm formulation of embodiment 1, wherein the formulation comprises: from about 1% to about 18% (w/w) of menthol; from about 3% to about 14% (w/w) of camphor; one or more moisturizers; one or more skin conditioning agents; and from about 0.1% to about 10% (w/w) of a plasticizer.
Embodiment 8. The massaging balm formulation of embodiment 7, wherein the formulation comprises: from about 1% to about 16% (w/w) of menthol; from about 3% to about 11% (w/w) of camphor; one or more moisturizers; one or more skin conditioning agents; and from about 0.1% to about 10% (w/w) of a plasticizer.
Embodiment 9. The massaging balm formulation of embodiment 7 wherein the formulation comprises: from about 14% to about 18% (w/w) of menthol; from about 8% to about 14% (w/w) of camphor; one or more moisturizers; one or more skin conditioning agents; one or more fragrances; and from about 0.75% to about 2% (w/w) of a plasticizer.
Embodiment 10. The massaging balm formulation of any one of embodiments 1 to 9, wherein the plasticizer is a pliable polyester wax.
Embodiment 11. The massaging balm formulation of embodiment 10, wherein the pliable polyester wax is synthetic beeswax.
Embodiment 12. The massaging balm formulation of any one of embodiments 7 to 11, wherein the one or more moisturizers and skin conditioners is selected from the group consisting of sweet almond (Prunus amygdalus dulcis) oil, sunflower (Helianthus annuus) seed wax, carnauba wax, cocoa (Theobroma cacao) seed butter, coconut oil (Cocos nucifera), avocado (Persea gratissima) oil or butter, and combinations thereof.
Embodiment 13. The massaging balm formulation of any one of embodiments 7 to 11, wherein the one or more moisturizers and skin conditioners comprises sweet almond (Prunus amygdalus dulcis) oil, sunflower (Helianthus annuus) seed wax, carnauba wax, cocoa (Theobroma cacao) seed butter, coconut oil (Cocos nucifera), and avocado (Persea gratissima) oil or butter.
Embodiment 14. The massaging balm formulation of any one of embodiments 9 to 11, wherein the one or more fragrances is selected from the group consisting of eucalyptus (Eucalyptus globulus) oil, grapefruit (Citrus paradisi) peel oil, peppermint (Mentha piperita) oil, spearmint (Mentha viridis) oil, and combinations thereof.
Embodiment 15. The massaging balm formulation of any one of embodiments 9 to 11, wherein the one or more fragrances comprises eucalyptus (Eucalyptus globulus) oil, grapefruit (Citrus paradisi) peel oil, peppermint (Mentha piperita) oil, and spearmint (Mentha viridis) oil.
Embodiment 16. The massaging balm formulation of any one of embodiments 7 to 15, wherein the balm formulation comprises: from about 14% to about 18% (w/w) of menthol; from about 8% to about 14% (w/w) of camphor; sweet almond (Prunus amygdalus dulcis) oil; sunflower (Helianthus annuus) seed wax; cocoa (Theobroma cacao) seed butter; coconut oil (Cocos nucifera); avocado (Persea gratissima) oil or butter; carnauba (Copemica cerifera) wax; eucalyptus (Eucalyptus globulus) oil; grapefruit (Citrus paradisi) peel oil; peppermint (Mentha piperita) oil; spearmint (Mentha viridis) oil; and from about 0.75% to about 2% (w/w) of a plasticizer.
Embodiment 17. The massaging balm formulation of embodiment 16, wherein the balm formulation comprises: from about 14% to about 18% (w/w) of menthol; from about 8% to about 14% (w/w) of camphor; from about 20% to about 30% (w/w) of sweet almond (Prunus amygdalus dulcis) oil; from about 10% to about 20% (w/w) sunflower (Helianthus annuus) seed wax; from about 8% to about 16% (w/w) cocoa (Theobroma cacao) seed butter; from about 7% to about 15% (w/w) coconut oil (Cocos nucifera); from about 1% to about 6% (w/w) avocado (Persea gratissima) oil or from about 3% to about 10% (w/w) avocado (Persea gratissima) butter; from about 0.5% to about 2.5% (w/w) carnauba (Copemica cerifera) wax; from about 0.01% to about 1% (w/w) eucalyptus (Eucalyptus globulus) oil; from about 0.01% to about 1% (w/w) grapefruit (Citrus paradisi) peel oil; from about 0.01% to about 1% (w/w) peppermint (Mentha piperita) oil; from about 0.01% to about 1% (w/w) spearmint (Mentha viridis) oil; and from about 0.75% to about 2% (w/w) of a plasticizer.
Embodiment 18. The massaging balm formulation of embodiment 16, wherein the balm formulation consists of: from about 14% to about 18% (w/w) of menthol; from about 8% to about 14% (w/w) of camphor; from about 20% to about 30% (w/w) of sweet almond (Prunus amygdalus dulcis) oil; from about 10% to about 20% (w/w) sunflower (Helianthus annuus) seed wax; from about 8% to about 16% (w/w) cocoa (Theobroma cacao) seed butter; from about 7% to about 15% (w/w) coconut oil (Cocos nucifera); from about 1% to about 6% (w/w) avocado (Persea gratissima) oil or from about 3% to about 10% (w/w) avocado (Persea gratissima) butter; from about 0.5% to about 2.5% (w/w) carnauba (Copemica cerifera) wax; from about 0.01% to about 0.5% (w/w) eucalyptus (Eucalyptus globulus) oil; from about 0.01% to about 0.5% (w/w) grapefruit (Citrus paradisi) peel oil; from about 0.01% to about 0.5% (w/w) peppermint (Mentha piperita) oil; from about 0.01% to about 0.5% (w/w) spearmint (Mentha viridis) oil; and from about 0.75% to about 2% (w/w) of a plasticizer.
Embodiment 19. A kit comprising the massaging balm formulation of any one of embodiments 1 to 18 and an applicator.
Embodiment 20. The kit of embodiment 19, wherein the kit comprises an applicator and/or instructions for use.
Embodiment 21. An applicator comprising a formulation of any one of embodiments 1 to 17, wherein the applicator has a surface in contact with the formulation.
Embodiment 22. The applicator according to embodiment 21, wherein the applicator is hand held.
Embodiment 23. The applicator according to embodiment 21, wherein the applicator has a surface in contact with the formulation and another surface configured for hand-holding the applicator.
Embodiment 24. The applicator according to any one of embodiments 21 to 23, wherein the applicator is approximately circular and wherein the applicator comprises a grid in contact with the formulation.
Embodiment 25. The applicator according to embodiment 23, wherein the grid is concave.
Embodiment 26. The applicator according to any one of embodiments 21 to 25, wherein the formulation on the applicator is shaped as a semi-sphere.
Embodiment 27. A method of generating an applicator comprising a formulation of any one of embodiments 1 to 18 comprising: providing an applicator having a top and bottom surface; and contacting the formulation with the top surface of the applicator, wherein the bottom surface of the applicator is configured for hand-holding of the applicator.
Embodiment 28. The method of embodiment 27, wherein the contacting comprises hot melting the formulation into a forming cup.
Embodiment 29. The method of embodiments 27 or 28 further comprising shaping the formulation into a desired shape.
Embodiment 30. The method of embodiment 27, wherein the method comprise shaping the formulation into approximately a semi-sphere.
Embodiment 31. The method of embodiments 29 or 30, wherein the shaping comprises use of a forming cup.
Embodiment 32. The method of embodiment 31, wherein the method further comprises use of a release agent to release the formulation from the forming cup.
Embodiment 33. The method of any one of embodiments 24 to 29, wherein applicator comprises a grip.
Embodiment 34. The method of any one of embodiments 24 to 33, wherein the top surface of the applicator is concave.
Embodiment 35. Use of the formulation of any one of embodiments 1 to 18 or an applicator of any one of embodiments 21 to 26 to treat pain and/or relieve itch.
Embodiment 36. A method of treating pain and/or relieving itch comprising contacting skin of a patient in need thereof with a formulation of any one of embodiments 1 to 18 or an applicator of any one of embodiments 22 to 27.

While the invention has been described and illustrated herein by references to various specific materials, procedures, and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1. A massaging balm formulation comprising:
a pain or itch relieving active selected from the group consisting of menthol, camphor, methyl salicylate, lidocaine, capsaicin, diphenhydramine, hydrocortisone, or combinations thereof; and
from about 0.1% to about 10% of a plasticizer.

2. The massaging balm formulation of claim 1 further comprising an essential oil.

3. The massaging balm formulation of claim 2, wherein the essential oil is lavender oil.

4. The massaging balm formulation of claim 1, wherein the pain or itch relieving active is menthol and camphor.

5. The massaging balm formulation of claim 1, wherein the pain or itch relieving active is menthol.

6. The massaging balm formulation of claim 1 wherein the formulation comprises about 1% to about 16% (w/w) of menthol; from about 3% to about 11% (w/w) of camphor, from about 62% to about 95.5% (w/w) of a plurality of waxes; from about 0.1% to about 10% (w/w) of a plasticizer; from about 0% to 2% (w/w) of essential oils.

7. The massaging balm formulation of claim 1, wherein the formulation comprises:
from about 1% to about 18% (w/w) of menthol;
from about 3% to about 14% (w/w) of camphor;
one or more moisturizers;
one or more skin conditioning agents; and
from about 0.1% to about 10% (w/w) of a plasticizer.

8. The massaging balm formulation of claim 7, wherein the formulation comprises:
from about 1% to about 16% (w/w) of menthol;
from about 3% to about 11% (w/w) of camphor;
one or more moisturizers;
one or more skin conditioning agents; and
from about 0.1% to about 10% (w/w) of a plasticizer.

9. The massaging balm formulation of claim 7 wherein the formulation comprises:
from about 14% to about 18% (w/w) of menthol;
from about 8% to about 14% (w/w) of camphor;
one or more moisturizers;
one or more skin conditioning agents;
one or more fragrances; and
from about 0.75% to about 2% (w/w) of a plasticizer.

10. The massaging balm formulation of claim 1, wherein the plasticizer is a pliable polyester wax.

11. The massaging balm formulation of claim 10, wherein the pliable polyester wax is synthetic beeswax.

12. The massaging balm formulation of claim 1, wherein the one or more moisturizers and skin conditioners is selected from the group consisting of sweet almond (Prunus amygdalus dulcis) oil, sunflower (Helianthus annuus) seed wax, carnauba wax, cocoa (Theobroma cacao) seed butter, coconut oil (Cocos nucifera), avocado (Persea gratissima) oil or butter, and combinations thereof.

13. The massaging balm formulation of claim 1, wherein the one or more moisturizers and skin conditioners comprises sweet almond (Prunus amygdalus dulcis) oil, sunflower (Helianthus annuus) seed wax, carnauba wax, cocoa (Theobroma cacao) seed butter, coconut oil (Cocos nucifera), and avocado (Persea gratissima) oil or butter.

14. The massaging balm formulation of claim 1, wherein the one or more fragrances is selected from the group consisting of eucalyptus (Eucalyptus globulus) oil, grapefruit (Citrus paradisi) peel oil, peppermint (Mentha piperita) oil, spearmint (Mentha viridis) oil, and combinations thereof.

15. The massaging balm formulation of claim 1, wherein the one or more fragrances comprises eucalyptus (Eucalyptus globulus) oil, grapefruit (Citrus paradisi) peel oil, peppermint (Mentha piperita) oil, and spearmint (Mentha viridis) oil.

16. The massaging balm formulation of claim 7, wherein the balm formulation comprises:
from about 14% to about 18% (w/w) of menthol;
from about 8% to about 14% (w/w) of camphor;
sweet almond (Prunus amygdalus dulcis) oil;
sunflower (Helianthus annuus) seed wax;
cocoa (Theobroma cacao) seed butter;
coconut oil (Cocos nucifera);
avocado (Persea gratissima) oil or butter;
carnauba (Copemica cerifera) wax;
eucalyptus (Eucalyptus globulus) oil;
grapefruit (Citrus paradisi) peel oil
peppermint (Mentha piperita) oil;
spearmint (Mentha viridis) oil; and
from about 0.75% to about 2% (w/w) of a plasticizer.

17. The massaging balm formulation of claim 16, wherein the balm formulation comprises:
from about 14% to about 18% (w/w) of menthol;
from about 8% to about 14% (w/w) of camphor;
from about 20% to about 30% (w/w) of sweet almond (Prunus amygdalus dulcis) oil;
from about 10% to about 20% (w/w) sunflower (Helianthus annuus) seed wax;
from about 8% to about 16% (w/w) cocoa (Theobroma cacao) seed butter;
from about 7% to about 15% (w/w) coconut oil (Cocos nucifera);
from about 1% to about 6% (w/w) avocado (Persea gratissima) oil or from about 3% to about 10% (w/w) avocado (Persea gratissima) butter;
from about 0.5% to about 2.5% (w/w) carnauba (Copemica cerifera) wax;
from about 0.01% to about 1% (w/w) eucalyptus (Eucalyptus globulus) oil;
from about 0.01% to about 1% (w/w) grapefruit (Citrus paradisi) peel oil;
from about 0.01% to about 1% (w/w) peppermint (Mentha piperita) oil;
from about 0.01% to about 1% (w/w) spearmint (Mentha viridis) oil; and
from about 0.75% to about 2% (w/w) of a plasticizer.

18. The massaging balm formulation of claim 16, wherein the balm formulation consists of:
from about 14% to about 18% (w/w) of menthol;
from about 8% to about 14% (w/w) of camphor;
from about 20% to about 30% (w/w) of sweet almond (Prunus amygdalus dulcis) oil;
from about 10% to about 20% (w/w) sunflower (Helianthus annuus) seed wax;
from about 8% to about 16% (w/w) cocoa (Theobroma cacao) seed butter;
from about 7% to about 15% (w/w) coconut oil (Cocos nucifera);
from about 1% to about 6% (w/w) avocado (Persea gratissima) oil or from about 3% to about 10% (w/w) avocado (Persea gratissima) butter;
from about 0.5% to about 2.5% (w/w) carnauba (Copemica cerifera) wax;
from about 0.01% to about 0.5% (w/w) eucalyptus (Eucalyptus globulus) oil;
from about 0.01% to about 0.5% (w/w) grapefruit (Citrus paradisi) peel oil;
from about 0.01% to about 0.5% (w/w) peppermint (Mentha piperita) oil;
from about 0.01% to about 0.5% (w/w) spearmint (Mentha viridis) oil; and
from about 0.75% to about 2% (w/w) of a plasticizer.

19. A kit comprising the massaging balm formulation of claim 1 and an applicator.

20. The kit of claim 19, wherein the kit comprises an applicator and/or instructions for use.

21. An applicator comprising a formulation of claim 1, wherein the applicator has a surface in contact with the formulation.

22. The applicator according to claim 21, wherein the applicator is hand held.

23. The applicator according to claim 21, wherein the applicator has a surface in contact with the formulation and another surface configured for hand-holding the applicator.

24. The applicator according to claim 21, wherein the applicator is approximately circular and wherein the applicator comprises a grid in contact with the formulation.

25. The applicator according to claim 23, wherein the grid is concave.

26. The applicator according to claim 21, wherein the formulation on the applicator is shaped as a semi-sphere.

27. A method of generating an applicator comprising a formulation of claim 1 comprising:
providing an applicator having a top and bottom surface; and
contacting the formulation with the top surface of the applicator,
wherein the bottom surface of the applicator is configured for hand-holding of the applicator.

28. The method of claim 27, wherein the contacting comprises hot melting the formulation into a forming cup.

29. The method of claim 27 further comprising shaping the formulation into a desired shape.

30. The method of claim 27, wherein the method comprise shaping the formulation into approximately a semi-sphere.

31. The method of claim 29, wherein the shaping comprises use of a forming cup.

32. The method of claim 31, wherein the method further comprises use of a release agent to release the formulation from the forming cup.

33. The method of claim 27, wherein applicator comprises a grip.

34. The method of claim 27, wherein the top surface of the applicator is concave.

35. (canceled)

36. A method of treating pain and/or relieving itch comprising contacting skin of a patient in need thereof with a formulation of claim 1 or a hand held applicator comprising the formulation of claim 1.
